(19) United States
(12) Patent Application Publication
(10) Pub. No.: US 2002/0044971 A1
(43) Pub. Date: Apr. 18, 2002

(54) Dissolution rate of poorly soluble drugs

(76) Inventors: Gordon L. Amidon, Ann Arbor, MI (US); John R. Crison, Ann Arbor, MI (US)

Correspondence Address: Gifford, Krass, Groh, Sprinkle,, Anderson & Citkowski, P.C., Suite 400, 280 N. Old Woodward, Birmingham, MI, 48009, US

(21) Appl. No.: 09/901,214

(22) Filed: Jul. 9, 2001

Related U.S. Application Data
(60) Provisional application No. 60/216,562, filed on Jul. 7, 2000.

(51) Int. Cl. A61K 9/14

(52) U.S. Cl. 424/484; 424/489

(57) ABSTRACT

A pharmaceutical delivery vehicle includes a solid drug particle disposed within a diffusional boundary layer made up of a matrix and a solubilizing agent. The solubilizing agent is selected to substantially solubilize a drug particle in vitro.

### RELATED APPLICATION

This application claims priority of U.S. Provisional Patent Application 60/216,562 filed Jul. 7, 2000 and is incorporated herein by reference.

### TECHNICAL FIELD

The present invention generally relates to a pharmaceutical excipient coating. More particularly, the present invention relates to a pharmaceutical excipient coating and method of making pharmaceutical compositions.

### BACKGROUND OF THE INVENTION

It is well known in the art that there are solid drugs which are scarcely soluble in water. Due to their low solubilities, these drugs have a correspondingly low degree of bioavailability.

The fundamental equation describing dissolution is:

$$\frac{ⅆ m}{ⅆ t} = \frac{SA \cdot {DC}_{sat}}{δ} \qquad Equation 1$$

where m is mass, t is time, D is the diffusion coefficient of the solid, Cₛₐₜ is the concentration of the drug at the solid-liquid interface, SA is the surface area available for dissolution, and δ is the thickness of the diffusional boundary layer. Based on equation 1, the critical determinants that control the dissolution rate are the diffusivity, concentration at the solid-liquid interface and the magnitude of the diffusional boundary layer. In most cases, the diffusional boundary layer does not change drastically from compound to compound, i.e., the range of diffusion coefficients is generally from 5×10⁻⁶ to 10×10⁻⁶ cm²/sec. Therefore, the drug concentration, surface area, and diffusional boundary layer are the key parameters that can be modified to improve the dissolution rate of poorly soluble compounds.

Methods used in the industry to improve the dissolution rate of poorly soluble drugs include:

1) micronization and milling to increase the surface area,

2) addition of surfactants to increase the concentration of drug and solid-liquid interface, and

3) mechanical energy to reduce the diffusional boundary layer.

However, these methods are difficult to put into practice due to the inherent physical chemical properties of the drugs as well as the physiological conditions present in the gastrointestinal (GI) tract. For example, micronization increases the surface area available for dissolution, however it also increases the change in free energy of the system when exposed to an aqueous solution. This results in particle aggregation and decreases the dissolution rate. The addition of surfactants to the system increases the solubility of the drug through micelle-facilitated dissolution, however, the large volume of fluid in the GI tract requires high concentrations of surfactants. This high concentration of surfactants can be toxic and damaging to the intestinal mucosa. Finally, increasing the mechanical energy of the system to reduce the diffusional boundary layer is useful for in vitro dissolution testing, however very impractical or impossible when applied to the in vivo system.

Several prior art processes have been developed in efforts to increase the solubility and, hence, the bioavailability of poorly soluble pharmaceuticals or drugs. One such prior art process disclosed in U.S. Pat. No. 5,851,275, to the present inventors, teaches the use of a gelatin and lecithin coating to prevent particle aggregation and thereby improve the dissolution rate of the drug. However, this patent does not teach controlling the parameters of the boundary layer as a means for increasing the dissolution rate of poorly water soluble drugs.

Therefore, it would be advantageous and desirable to have a method of increasing the dissolution rate of poorly water-soluble pharmaceuticals which is an advance over the prior art methods. It would be a further advantage to have a coating and method of coating a drug which is not affected by gastric pH, can be applied to a drug in aqueous solution using standard manufacturing and equipment for coating the drug, and which is safe and not destructive to the intestinal mucosa.

By combining the method and coating of the present invention with poorly water-soluble drugs or pharmaceutical compositions, optimal advantage can be taken of the potential potency and efficacy of poorly water-soluble drugs by increasing their bioavailability. The present invention provides an improved method and coating for controlling the characteristics and properties of the diffusional boundary layer disposed about a drug particle which allows for greater dissolution rate and, hence, greater bioavailability.

### SUMMARY OF THE INVENTION

A pharmaceutical delivery vehicle includes a solid drug particle disposed within a diffusional boundary layer made up of a matrix and a solubilizing agent. The solubilizing agent is selected to substantially solubilize a drug particle in vitro.

### BRIEF DESCRIPTION OF THE INVENTION

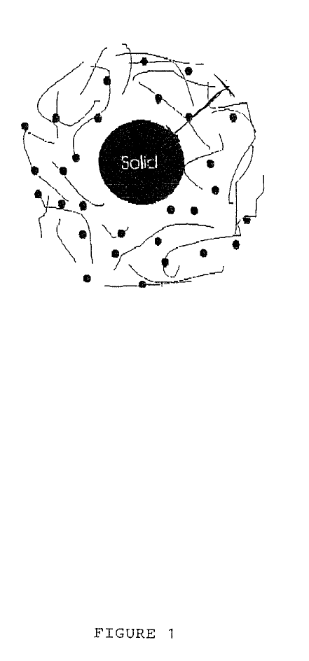

FIG. 1 is a schematic showing a solid inventive drug particle within a boundary layer of a matrix component and a surfactant;

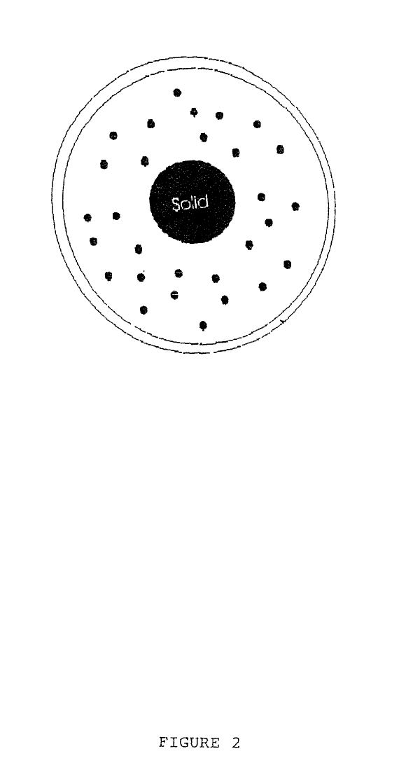

FIG. 2 is a schematic showing a solid inventive drug particle within a boundary layer of a matrix component and a continuous envelope;

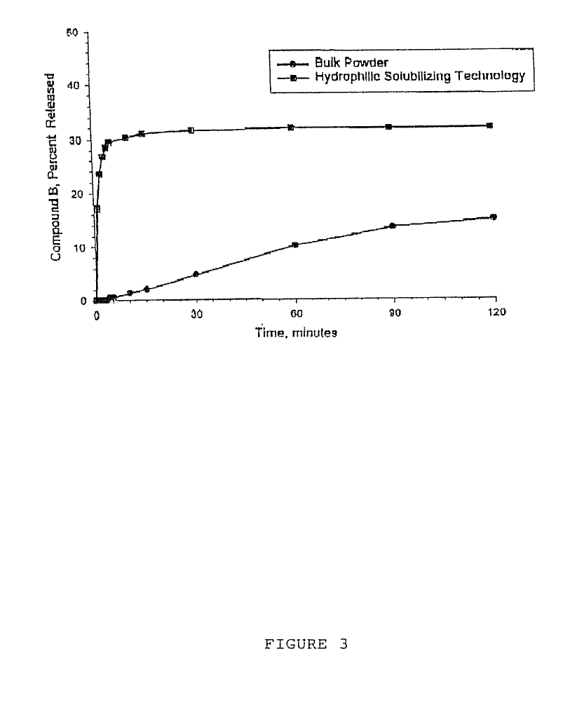

FIG. 3 is a graph illustrating release of compound B as a function of time according to the instant invention as compared to bulk powder; and

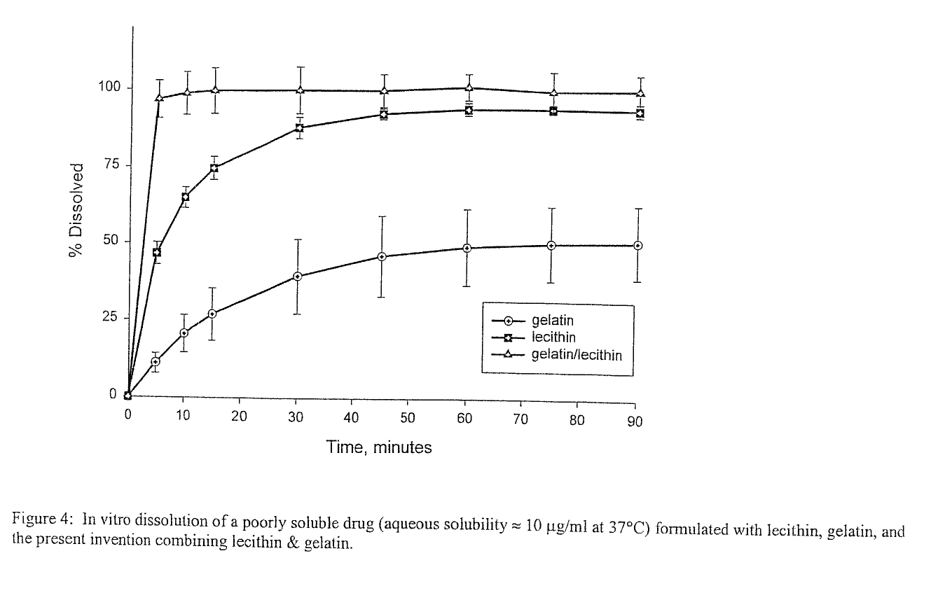

FIG. 4 is a graph showing the percent dissolution of a poorly soluble drug according to the present invention formulated by combining lecithin and gelatin. Comparative dissolution for lecithin alone and gelatin alone.

### DETAILED DESCRIPTION IF THE INVENTION

The present invention involves controlling the thickness of the diffusional boundary layer and controlling the concentration of the drug at the solid-liquid interface. The size of the diffusional boundary layer is maintained at a volume sufficient to solubilize substantially the entire drug particle. A surfactant or emulsion/microemulsion system is included to enhance the solubility of the drug and reduce the volume requirements of the boundary layer. The boundary layer may be comprised of a matrix component such as a polymer embedded with the surfactant, or a film or envelope surrounding a drug particle and containing a surfactant or emulsion/microemulsion system (see FIGS. 1 and 2).

In other words, the dissolution rate can be enhanced by maintaining a region adjacent to the drug particle which contains sufficient surfactant micelles to substantially solubilize the drug particle without increasing the surfactant concentration in the bulk fluid of the GI tract. The size of the diffusional boundary layer can be controlled, i.e., made larger or smaller, by selecting the components forming the boundary layer which have a known hydrated volume. Additionally, the concentration of a particular surfactant use with a particular drug can be calculated using an equilibrium coefficient which is based on the solubilizing power of the surfactant and its relationship to a particular drug or compound.

The volume requirement of the boundary layer is a function of the solubilizing effect of the surfactant. This volume can be calculated by dividing the mass of the particle by the solubility of the drug.

For example:

$$\frac{{M}_{p}}{{C}_{sat}} = {V}_{BL} \qquad Equation 2$$

where Mₚ is the mass of the particle, Cₛₐₜ, is the solubility, and V_{BL} is the volume of the boundary layer.

More than one pharmaceutical ingredient at a time can be treated according to the present invention to yield a desired pharmaceutical composition. Additionally, poorly water-soluble pharmaceutical ingredients can be treated according to the present invention and can then be used in combination with other pharmaceutical ingredients which therefore may or may not be poorly water-soluble.

The term “pharmaceutical ingredient” includes any pharmaceutical compound, drug, or composition in solid form such as powder or granules.

The method generally includes the steps of solubilizing the matrix forming component in water heated. The surfactant component is added to the matrix component/water mixture and is thoroughly mixed therein. At least one pharmaceutical ingredient or drug in solid particulate form is then added slowly and mixed so as to cause thorough and uniform coating of the particles of the pharmaceutical ingredient. Following coating with the matrix/surfactant mixture, the coated pharmaceutical ingredient is then dried.

The coating or application step can be accomplished by simple immersion of the particles of the pharmaceutical ingredient in the matrix/surfactant mixture.

After the pharmaceutical ingredient(s) is coated with the aqueous mixture of the matrix component and the surfactant component, the aqueous solvent water can be removed by various techniques.

The solvent removal or drying of the coated pharmaceutical ingredient can be accomplished by lyophilization or freeze drying of the coated particles by techniques known to those skilled in the art. Lyophilization, or freeze-drying, is a process by which a solid is dissolved or suspended in a liquid, frozen and the water is sublimed from the product after it is frozen. The advantage of this process is that the stability of biologicals and pharmaceuticals that are unstable in the presence of water can be increased without elevated temperatures that often occur during processing. (Avis, 1975).

The coated pharmaceutical ingredient can also be dried by the method known in the art as spray drying. Spray drying and fluidized bed processing are widely used in the industry for drying, granulating and coating active ingredients (drugs) (Jones, 1991). These methods enable the pharmaceutical formulator to convert solid drug particles into powders and granulations with excellent flow and compression properties for high speed manufacturing of tablets and capsules. The basic design consists of a spray nozzle, a drying chamber, and an air source. The drug, along with other solubilized or suspended materials, is pumped through a spray nozzle, atomized and dried into a fine, amorphous powder. Alternatively, it is coated onto sugar seeds (non-pareils) or dried into aggregates. The spraying rate, air flow and temperature of the drying chamber all can be varied to produce the desired end product. This process is widely used in the pharmaceutical industry and the invention described in this patent has been shown to be manufacturable by this method.

The coated pharmaceutical ingredient can also be granulated to obtain granules having good redispersability in water with granule diameters in the range of 4 to 1000 microns. The granulation can be accomplished using a fluid bed granulator (Glatt( Ramsey, N.J.) using means well known to those skilled in the art.

Additionally, the method of the present invention can include the step of spray coating the matrix/surfactant coated pharmaceutical ingredient onto micronized particles. Micronization is the process by which solid drug particles are reduced in size as is well known in the art. Since the dissolution rate is directly proportional to the surface area of the solid, and reducing the particle size increases the surface area, reducing the particle size increases the dissolution rate.

The theoretical basis for using micronization to increase the dissolution rate is as follows: Drug dissolution is defined by equation 1:

$$\frac{ⅆ m}{ⅆ t} = \frac{SA \cdot D}{h} \cdot ( {C}_{s} - {C}_{b} ) \qquad ( 1 )$$

where m is the mass of drug, t is time, SA is surface area, C_{S} is the solubility of the drug, h is the diffusional boundary layer thickness, and C_{b} is the concentration of drug in the bulk solution. Applying equation 1 to a spherical particle and assuming sink conditions:

$$\frac{ⅆ r}{ⅆ t} = \frac{D}{hρ} \cdot {C}_{s} \qquad ( 2 )$$

Furthermore, if we integrate equation 2 and assume the diffusional boundary layer to be equal to the radius of the particle, then the mass of drug dissolved for a given period of time is inversely proportional to the square of the radius of the particle.

$${\int }_{{r}_{n}}^{r} r ⅆ r = \frac{D \cdot {C}_{s}}{hρ} {\int }_{0}^{r} ⅆ t \\ r - {r}_{0} = \frac{D \cdot {C}_{s}}{hρ} t \\ {M}_{0}^{1 / 3} - {M}_{}^{1 / 3} = \frac{{M}_{0}^{1 / 3}}{2 r} \cdot \frac{2 D \cdot {C}_{s}}{rρ} t \\ \% D = ( 1 - \frac{D \cdot {C}_{s}}{{r}^{2} ρ} t )$$

$$where \% D is the percent of drug dissolved .$$

Micronizing equipment typically create particles with diameters in the micron range (1 to 20 μm). However, there are drawbacks to micronizing water insoluble drugs. Since most water insoluble drugs have very high surface energy, as the surface area increases, so does the surface energy and micronized particles generally aggregate to reduce the amount of free energy resulting in particles larger than prior to micronization (see Figure below).

For example:

ΔG=γ·SA=γ·πr²

where ΔG is the free energy, γ is the surface tension of the solid and r is the particle radius. Surface active materials, such as in the invention presented here, reduce the surface tension of the solid, lower the free energy of the system and eliminate particle aggregation. Examples of commercially available micronizers are Fluid Energy Aljet (Plumsteadville, Pa.) and Sturtevant, Inc. (Boston, Mass.).

Although micronization results in increased surface area causing particle aggregation, which can negate the benefit of micronization and is an expensive manufacturing step, it does have the significant benefit of increasing the dissolution rate of water insoluble drugs if particle aggregation can be prevented.

The active pharmaceutical ingredient utilized in the method of the present invention can include, for example, griseofulvin, cyclosporin (see Table 1 for aqueous solubilities of these compounds and other suitable pharmaceutical ingredients or drugs having low water solubility).

Other examples of water insoluble drugs that can benefit from the present invention are listed in Table 1. This list in Table 1 is not meant to be exhaustive, but rather as an exemplary list.

Applicant has conducted a dissolution study demonstrating the increased dissolution rate of water-insoluble pharmaceutical ingredients according to the present invention as shown in FIG. 3.

Theoretical considerations of drug dissolution and absorption in the human gastrointestinal tract indicate that for water insoluble drugs two independent variables will control drug absorption: the dissolution rate extent of dissolution and dose of drug given. The significance of this analysis is that for water insoluble drugs, the fraction dose absorbed is inversely proportional to dose and is directly proportional to the dissolution rate. Therefore, in vivo solubilization and dissolution are important determinants of drug absorption.

### Intestinal Drug Absorption-Theoretical Considerations Membrane Permeability and Luminal/Wall Concentration

The fundamental equation describing drug absorption is:

J_{W}=P_{W}·C_{W} equation 1

where, J_{W}(x,y,z,t) is the drug flux (mass/time/area) through the intestinal wall at any position and time, P_{W}(w,y,x,t) is the permeability ofthis (complex) membrane, and C_{W}(w,y,z,t) the drug concentration at the membrane (wall) surface (known as Ficks' First Law). This is Ficks' First Law applied to a membrane and applies at each point along the membrane (1) i.e. equation 1 is a local law pertaining to each point along the intestinal membrane. Equation (1) states that the critical parameters governing drug absorption are the intestinal permeability and the concentration of drug in solution at the intestinal surface. P_{W} here is assumed to be high since the drugs are lipophilic. Therefore the focus will be the term C_{W.}

### In Vivo Drug Dissolution and Luminal/Wall Concentration

The equation which describes the processes governing mass transport in the intestine (tube) is (15):

∂C/∂t+v·∇c=D∇²C+R equation 2

where, C is the local concentration, v is the local velocity, D the diffusivity, and R the rate of production of solute. This equation applies to all components in the intestinal fluid medium and, in general, is much too complex to solve. However, a simpler quantitative and predictive model for drug absorption based on this equation has been developed (Amidon et al., 1995; Crison and Amidon, 1995).

This model considers a segment of intestine over which the permeability may be considered constant, a plug flow fluid with the suspended particles as moving with the fluid, no significant particle-particle interactions (i.e. aggregation) and dissolution in the small particle limit, leading to the following pair of differential equations in dimensionless form:

dr/dz=−(Dn/3) C*(1−C*)/ r* equation 3

and

dC*/dz*=DoDn·r*(1−C*)−2AnC* equation4

where

z*=z/L=(ᵥ_{_{z}}/L(t=t*

t*=t/(L/ᵥ_{_{z}})=t/(AL/Q)=t/(V/Q)

where L=tube length, v_{z}=axial fluid velocity in the tube, A=tube surface area, area=2πRL, R=tube radius, Q=fluid rate=Av_{z}. The three important dimensionless groups are:

$$\begin{matrix}Do = Dose Number = \frac{{M}_{0} / {V}_{0}}{{C}_{3}} & equation 5 \\ Dn = Dissolution Number = \frac{{DC}_{3}}{{r}_{o}} \cdot \frac{4 π {r}_{o}^{2}}{\frac{4}{3} π {r}_{o}^{3} ρ} \cdot {t}_{res} = \frac{3 {DC}_{s}}{{r}_{n}^{2} ρ} \cdot {t}_{res} = \frac{{t}_{res}}{{t}_{Diss}} & equation 6 \\ An = Absorption Number = \frac{{P}_{eff}}{R} \cdot {t}_{res} = {t}_{abs}^{- 1} \cdot {t}_{res} \\ {t}_{res} = π {R}^{2} L / Q = mean tube residence time . \\ {t}_{Diss} = \frac{{r}_{o}^{2} ρ}{3 {DC}_{s}} = time required for a particle of the drug to dissolve . \\ {t}_{abs}^{-} 1 = {k}_{abs} = 2 \cdot \frac{{P}_{eff}}{R} = \\ the effective absorption rate constant . & equation 7\end{matrix}$$

Where, in addition to the symbols defined previously, M₀ is the dose, ro is the initial particle radius, C_{S} is the solubility, p is the density, P_{eff} is the effective permeability, tᵣₑₛ is the residence time, t_{abs} is the absorption time, and t_{diss} is the dissolution time (Oh et al., 1993).

The initial conditions for this set of differential equations are:

r=rₒt=0

C=Cₒt=0

It is convenient to define a more general initial condition for the concentration of drug entering the intestine using the initial saturation, is:

C_{S}(O)

C_{S}

where C_{S} is the solubility of the drug, and C_{S}(O) is the concentration of the drug entering the intestine.

As is evident from the above dimensional analysis of Equation 2, there are three dimensionless groups that describe the total dissolution and absorption process of drugs in the intestine: the dissolution number (DN), dose number (Do), and absorption number (An), or the dissolution rate, the dose of drug given, and the rate of absorption, respectively.

### Strategies for Improving the Dissolution

### and Absorption of Water Insoluble Drugs

The class of drugs that are being considered in this patent are those that have low solubility, i.e., drugs where the concentration greatly exceeds the solubility. The absorption of these drugs is limited by how much drug can get into solution. According to the Hixon-Crowell cube root law for estimating the dissolution of a powder, the percent of drug in solution in a given period of time is a function of the particle size and the solubility.

$$\% D = {[ \frac{{DC}_{s}}{{r}^{2} ρ} \cdot t ]}^{3} \times 100$$

where D, Cₛ, r, t and p are previously defined. In the case of water soluble drugs, the solubility, or Cₛ is low such that the dose of drug given exceeds the amount of fluids available in the GI tract for complete dissolution to take place. Therefore, reducing the particle size or increasing the solubility, or both, are methods for increasing the dissolution rate and absorption of a water insoluble drug.

There are disadvantages to these two methods. First, due to their high lipophilic nature, these drugs have very high surface energy and the interfacial tension between the solid and water is high. The free energy for particles in water is proportional to the interfacial tension and the surface area of the particle as follows:

ΔG=γSA

Where ΔG is the free energy of the system, γ is the interfacial tension between the liquid and solid, and SA is the surface area of the solid. Reducing the particle size increases the surface area resulting in an increase in the free energy. To lower the free energy, the parties aggregate and negate the utility of particle size reduction.

The second disadvantage comes from attempting to increase the solubility. Two methods can be used to increase the solubility of a drug, 1) formation of a salt, and 2) incorporating surfactants in the formulation. Salt formation has been used successfully but is limited to weak acids and bases. In theory, surfactants should work well to increase the solubility, however, the concentration of surfactants needed to overcome the dilution effect of the GI tract often exceeds safe levels. Since surfactants are surface active, effective concentrations can often disrupt biological membranes creating holes in the intestinal mucosa.

Examples of drugs currently on the market that fall into the category of water insoluble drugs are listed in Table 1. The volume of fluid required to achieve complete dissolution is given in column 4. Assuming that a dose of drug is normally administered with a glass of water, approximately 0.25 liters, it is clear that fluid requirements for complete dissolution greatly exceed the fluid initially available. The brevity of this list confirms the importance of a drug's solubility to achieving a successful, marketable product.

The significance of solubility and dissolution rate to absorption are clearly defined in the dose and dissolution numbers. More precisely, the dissolution rate is important due to the limited residence time in the intestine for any given drug particle. The dependency of absorption on dose and dissolution is also noteworthy since it emphasizes that it is the dose number, rather than just the solubility, that needs to be included in predicting drug absorption. Other physical constants such as diffusivity and particle density contribute to the dissolution process, however, the range of values for these constants for most organic compounds is small. FIGS. 1-b are plots of the fraction of dose absorbed vs. dose and dissolution number as generated from equations 2-6. The surface presented in these Figures clearly shows that reduction of particle size is a valid method for increasing the amount of drug absorbed, provided the particles do not aggregate and form large clumps. Below are two examples illustrating this invention.

### EXAMPLES

### Experimental

The materials used in this example are gelatin for the polymeric boundary layer and lecithin as the surfactant for the micelles; however, it should be noted that the gelatin and lecithin are merely exemplary of the components which could be used in the present invention and are not meant to be limiting. Other suitable boundary layer forming components and surfactants are contemplated by the present invention. The drug is incorporated into this system by:

1) Solubilizing the gelatin in water.

2) Suspending the lecithin in water.

3) Mixing the two together.

4) Adding the drug, and then mixing well until the solid particles are dispersed.

5) Removing the water using lyophilization, spray drying, fluid bed technologies, etc.

When the particles rehydrate, a gelatin polymeric matrix with lecithin micelles embedded exists. The drug then solubilizes within this matrix (see FIG. 3).

The results of the above studies demonstrate that the lecithin/gelatin coating of the present invention greatly increases both the initial dissolution rate and total percentage dissolution of previously poorly water-soluble pharmaceutical ingredients. That is, the method of coating a poorly water-soluble pharmaceutical ingredient with a pharmaceutical excipient or coating formulation which includes lecithin and gelatin greatly increased both the initial dissolution rate and overall percent dissolution of the previously poorly water-soluble pharmaceutical ingredient. The increased dissolution of the pharmaceutical ingredients treated according to the present invention allows drugs which may have poor water-solubility to be utilized since the method and coating of the present invention greatly increases the dissolution rate of these poorly water-soluble pharmaceuticals contained therein.

Pharmaceutical ingredients prepared according to the method of the present invention can be formed into tablets or loaded into capsules by methods well known to those skilled in the art without losing their enhanced dissolution rate in aqueous solution. The present invention has been shown to function in vitro as well as in vivo.

Throughout this application various publications and patents are referenced by citation or number. Full citations for the publications are listed below. The disclosure of these publications or patents in their entireties are hereby incorporated by reference into this application in order to more fully describe the state of the art to which this invention pertains.

The invention has been described in an illustrative manner, and it is to be understood the terminology used is intended to be in the nature of description rather than of limitation.

Obviously, many modifications and variations of the present invention are possible in light of the above teachings. Therefore, it is to be understood that within the scope of the appended claims, the invention may be practiced otherwise than as specifically described.

TABLE 1
Examples of Currently Marketed Water Insoluble Drugs

| | Solubility | | |
|---|---|---|---|
| Formulation | (mg/ml) | Dose (mg) | Y_{DISSOLUTION} (liters) |
| Cyclosporin¹¹ | 0.006 | 750 | 125 |
| Griseofulvin¹² | 0.017 | 500 | 29.4 |
| Digoxin¹³ | 0.024 | 0.50 | 0.021 |
| Nifedipine¹⁴ | 0.010 | 30.0 | 3.0 |
| Itraconozole¹⁵ | 0.001 | 200 | 9.9 |
| Carbamazepine¹⁶ | 0.400 | 200 | 4.3 |
| Piroxicam¹⁵ | 0.010 | 20.0 | 8.2 |
| Fluconazole¹³ | 0.100 | 200 | 2.0 |
| Finasteride¹³ | 0.001 | 5.00 | 5.0 |
| Diflumisal¹³ | 0.010 | 1000 | 3.6 |

¹¹J. P. Reymond, J. L. Steimer, and W. Niederberger, J Pharmacokinet, Biophar. 16:331-353 (1988).
¹²B Katchen, S. J. Symchowicz, J. Pharm. Sci. 56:1108 (1967). note: solubility was measured at 39° C.
¹³J. B. Dressman, D. Fleisher, Mixing-tank Model for Predicting Dissolution Rate Control of Oral Absorption, J. Pharm. Sci. 75:109-116 (1986).
¹⁴D. R. Swanson et al., Nifedipine Gastrointestinal Therapeutic System, Amer. J. of Med. 83:3-9 (1987).
¹⁵The Merck Index, Eleventh Edition, Merck & Co., Inc., Rahway, N.J., 1989.
¹⁶J. R. Crison, Estimating the Dissolution and Absorption of Water Insoluble Drugs in the Small Intestine, Ph.D. Thesis, The University of Michigan, 1993.

1. A pharmaceutical delivery vehicle, said delivery vehicle comprising:
a drug particle disposed within a diffusional boundary layer comprising a matrix and a solubilizing agent;
said matrix and said solubilizing agent forming the diffusional boundary layer, wherein said solubilizing agent is capable of substantially solubilizing said drug particle.

2. A delivery vehicle according to claim 1, wherein said solubilizing agent comprises a surfactant.

3. A delivery vehicle according to claim 1, wherein said solubilizing agent comprises an emulsion.

4. A delivery vehicle according to claim 3, wherein said emulsion comprises a micro emulsion.

5. A delivery vehicle according to claim 1, wherein said solubilizing agent comprises lecithin.

6. A delivery vehicle according to claim 1, wherein said matrix comprises a polymer.

7. A delivery vehicle according to claim 1, wherein said matrix comprises a film.

8. A delivery vehicle according to claim 6, wherein said polymer comprises a carbohydrate.

9. A delivery vehicle according to claim 8, wherein said carbohydrate comprises gelatin.

10. A delivery vehicle according to claim 1, wherein said boundary layer comprises said matrix embedded with said solubilizing agent.

11. A delivery vehicle according to claim 1, wherein said boundary layer substantially encloses said drug particle and said solubilizing agent.

12. A pharmaceutical delivery vehicle, said delivery vehicle comprising:
a drug particle disposed within a diffusional boundary layer having volume sufficient to substantially solubilize said drug particle.

13. A delivery vehicle according to claim 12, wherein said diffusional boundary layer comprises a matrix.

14. A delivery vehicle according to claim 12, wherein said matrix includes a solubilizing agent disposed therein.
